(19) United States
(12) Patent Application Publication
(10) Pub. No.: US 2006/0223072 A1
(43) Pub. Date: Oct. 5, 2006

(54) Methods of using a DNase I-like enzyme

(76) Inventors: Barry E. Boyes, Wilmington, DE (US); Rhonda R. Taylor, Smyma, DE (US); John R. Link, Wilmington, DE (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 11/097,395

(22) Filed: Mar. 31, 2005

(51) Int. Cl. C12Q 1/68; C12P 19/34; C12N 9/22

(52) U.S. Cl. 435/6; 435/199; 435/91.2

(57) ABSTRACT

Methods for expanding conditions of use of a DNase I-like enzyme are disclosed as are compositions and kits comprising a DNAse I-like enzyme. Methods and kits for isolating RNA are also disclosed.

### BACKGROUND

Deoxyribonuclease I (DNase I) is an enzyme that is used in both research and clinical setting, e.g., in the treatment of cystic fibrosis (Ramsey, New Engl. J. Med. 1996; 335:179-188). The enzyme is a DNA endonuclease which catalyzes the hydrolysis of double-stranded DNA (dsDNA) by a double-strand or single-strand nick, leading to the depolymerization of DNA. The activity of the enzyme is maximal over a pH range of 6-9, dependent on the presence of divalaent cations, such as Ca⁺², Mg⁺², and Mn⁺² and is inhibited by the presence of monovalent salts such as NaCl and KCl. (Kunitz, J. Gen. Physiol. 1950:33:349-362; Campbell and Jackson, J. Biol. Chem. 1980;255:3726-3735 DNase I also is strongly inhibited by globular actin (G-actin) (Lazarides and Lindberg, Proc. Natl. Acad. Sci. USA 1974:71:4742-4746).

DNase I is often used as a reagent for the removal of residual or unwanted DNA from solutions of RNA, e.g., during the purification of RNA from biological sources. In addition, DNase I is used in techniques for generating synthetic RNA, such as in in vitro transcription reactions, for generating cRNA, and in the enzymatic cleavage of double-stranded DNA in DNA footprinting assays to detect protein binding sites.

In such applications, it is desirable to have the ability to employ DNase I activity under the broadest possible conditions of use, for example, under conditions that may normally be inhibitory to the native enzyme. For example, recombinant DNase I enzymes have been engineered that retain significant activity in the presence of higher concentrations of NaCl, permitting the efficient digest of DNA in solutions of this salt. See, e.g., U.S. Patent Publication 20040219529; Pan and Lazarus, J. Biol Chem. 1998;273:11701-08. Other recombinant enzymes have been generated which are resistant to inhibition by G-action. See, e.g., Pan, et al. J Biol Chem. 1998; 273(29):18374-81. An alternative approach to engineering novel enzymes is to compensate for decreased specific activity of DNase I by adding large quantities of a partially inhibited enzyme, thereby providing sufficient activity to digest DNA. See, e.g., as described in U.S. Pat. No. 6,218,531. This approach has certain practical disadvantages, including waste of a costly enzyme, an increased potential for carry-over of enzyme into downstream processes, as well as increased potential for adding contaminating RNase activity, which is frequently observed in DNase I preparations.

### SUMMARY OF THE INVENTION

The invention relates to methods for isolating RNA from a sample comprising both RNA and DNA through the use of DNase I under conditions which are normally inhibitory to the native enzyme as well of compositions and kits for facilitating the method. It is a discovery of the instant invention, that under these conditions, enhanced recovery of RNA is possible.

In one embodiment, the invention relates to a composition comprising a DNase I-like enzyme and an organic solvent, which is not glycerol, and an RNAse inhibitor. In one aspect, the organic solvent comprises an alcohol.

In certain embodiments, the organic solvent is present in at least about 20% v/v in an aqueous solution comprising the DNase I-like enzyme, or at least about 40%, at least about 60% or up to about 99% v/v organic solvent. In certain aspects, the aqueous solution comprises a solution which would be inhibitory to the DNase I-like enzyme in the absence of organic solvent.

In one aspect, the DNase I-like enzyme comprises bovine pancreatic DNase I. In another aspect, the DNase I-like enzymes comprises a recombinant enzyme.

In certain aspects, the alcohol comprises a monohydroxyl alcohol, such as, for example, methanol, ethanol, isopropanol, butanol, isomers thereof, stereoisomers thereof, and combinations thereof.

In other aspects, the alcohol comprises a di-hydroxylic alcohol, such as, for example, ethane diol, propane diol, butane diol, isomers thereof, stereoisomers thereof, and combinations thereof.

In still other aspects, the alcohol comprises a tri-hydroxylic alcohol.

In certain aspects, the alcohol comprises a combination of one or more different monohydroxyl alcohols, di-hydroxylic or tri-hydroxlic alcohols. Although, in one aspect, the composition comprises at least one non-glycerol alcohol, the composition may additionally include glycerol.

Suitable RNase inhibitors for use in the present invention include, but are not limited to, one or more of inhibitors of RNase A, RNase B, RNase C, RNase T1 and RNase 1.

The invention further relates to kits. In one aspect, the invention provides a kit comprising any of the compositions described above and an aqueous solution provided in a separate container from the composition, e.g., to alter the concentration of solvent relative to aqueous solution in the composition. In another aspect, the aqueous solution comprise a solution which would be inhibitory to the DNase I-like enzyme in the absence of organic solvent.

In certain aspects, the kit comprises an DNase I-like enzyme and an organic solvent in separate containers. In a further aspect, the kit further comprises an aqueous solution which is optionally, in a separate container from the organic solvent. In another aspect, the aqueous solution comprises a solution which would be inhibitory to the DNase I-like enzyme in the absence of organic solvent. In still another aspect, the kit comprises an RNase inhibitor provided in a separate container from the DNase I-like enzyme and organic solvent.

The invention also relates to a method that comprises contacting a sample comprising a DNA molecule and RNA molecule with a DNase I-like enzyme and a solution comprising an organic solvent which is not glycerol; and collecting the RNA molecule. In certain aspects, the solution comprises a salt concentration inhibitory to the DNase I-like enzyme in the absence of organic solvent. However, in other aspects, the solution does not comprise salt. In a further aspect, the solution comprises a potentiating amount of organic solvent which maximizes collection of RNA from the sample.

The organic solvent can comprise an alcohol, such as a monohydroxyl, di-hydroxylic or tri-hydroxylic alcohol or combinations of such alcohols as discussed above. In certain aspects, in addition to the organic solvents described above, glycerol can additionally be added.

The sample can be a cell or tissue sample or a partially purified nucleic acid sample.

In one aspect, the RNA molecule is collected by contacting the sample with an RNA capture material. In another aspect, the method further comprises releasing the RNA molecule from the RNA capture material. In a further aspect, the RNA capture material comprises a polymeric membrane.

In still another aspect, the method further comprises contacting the sample to a solid phase under conditions in which genomic DNA preferentially remains associated with the solid phase. In one aspect, contacting to the solid phase occurs prior to contacting the sample to the RNA capture material.

The invention also provides an RNA capture material comprising a solid phase in contact with a DNase I-like enzyme and an organic solvent which is not glycerol. In one aspect, the material comprises a polymeric membrane. In another aspect, the membrane comprises polysulfone.

### BRIEF DESCRIPTION OF FIGURES

FIG. 1 is a schematic diagram illustrating various steps of a process for isolating RNA using a DNase I-like enzyme according to one aspect of the invention.

FIG. 2 is a graph comparing the effects of different organic solvents on RNA recovery.

FIG. 3 illustrates the effect of alcohol additives during DNase I digestion on RNA Isolation in a BioAnalyzer 2100 RNA PicoAssay.

### DESCRIPTION OF THE INVENTION

Before describing the present invention in detail, it is to be understood that this invention is not limited to specific compositions, method steps, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” includes more than one biopolymer, and reference to “a voltage source” includes a plurality of voltage sources and the like.

Definitions

The following definitions are provided for specific terms that are used in the following written description.

The term “binding” refers to two molecules associating with each other to produce a stable composite structure under the conditions being evaluated (e.g., such as conditions suitable for RNA or DNA isolation). Such a stable composite structure may be referred to as a “binding complex”.

As used herein, the term “RNA” or “oligoribonucleotides” refers to a molecule having one or more ribonucleotides. The RNA can be single, double or multiple-stranded (e.g., comprise both single-stranded and double-stranded portions) and may comprise modified or unmodified nucleotides or non-nucleotides or various mixtures and combinations thereof.

As used herein, the term “DNA” or “deoxyribonucleotides” refers to a molecule comprising one or more deoxyribonucleotides. The DNA can be single, double or multiple-stranded (e.g., comprise both single-stranded, double-stranded, and triple-stranded portions) and may comprise modified or unmodified nucleotides or non-nucleotides or various mixtures and combinations thereof.

As used herein “complementary sequence” refers to a nucleic acid sequence that can form hydrogen bond(s) with another nucleic acid sequence by either traditional Watson-Crick or other non-traditional types (for example, Hoogsteen type) of base-paired interactions.

In certain embodiments, two complementary nucleic acids may be referred to as “specifically hybridizing” to one another. The terms “specifically hybridizing,” “hybridizing specifically to” and “specific hybridization” and “selectively hybridize to,” are used interchangeably and refer to the binding, duplexing, complexing or hybridizing of a nucleic acid molecule preferentially to a particular nucleotide sequence under stringent conditions. “Hybridizing” and “binding”, with respect to polynucleotides, are used interchangeably.

The term “reference” is used to refer to a known value or set of known values against which an observed value may be compared.

It will also be appreciated that throughout the present application, that words such as “cover”, “base” “front”, “back”, “top”, “upper”, and “lower” are used in a relative sense only.

As used herein, the term “solid phase” or “solid substrate” or “matrix” includes rigid and flexible solids. Examples of solid substrates include, but are not limited to, gels, fibers, whiskers, resins, microspheres, spheres, cubes, particles of other shapes, channels, microchannels, capillaries, walls of containers, membranes and filters.

As used herein, the term “silica-based” is used to describe SiO₂ compounds and related hydrated oxides and does not encompass silicon carbide compositions, which are described herein.

As used herein, a “nucleic acid binding material”, stably binds a nucleic acid (e.g., such as double-stranded, single-stranded, partially double-stranded, or triple-stranded DNA, RNA or modified form thereof). By “stably binds” it is meant that under defined binding conditions the equilibrium substantially favors binding over release of the subcellular component, and if the solid substrate containing a selected bound subcellular component is washed with buffer lacking the component under these defined binding conditions, substantially all the component remains bound. In particular embodiments the binding is reversible. As used herein, the term “reversible” means that under defined elution or release conditions the bound nucleic acid component of a sample is predominantly released from the nucleic acid binding material and can be recovered (e.g., in solution). In particular embodiments, at least about 10%, at least about 20%, at least about 50%, at least about 60%, at least 90%, or at least 95% of the bound nucleic acid component is released under the defined elution or release conditions.

As used herein, a “nucleic acid capture material” is one which preferably retains, traps, or remains associated with nucleic acids to remove a nucleic acid from solution. A nucleic acid capture material may, but does not necessarily bind to a nucleic acid molecule.

“Washing conditions” include conditions under which unbound or undesired components are removed from a module of a device described below.

The term “assessing” “inspecting” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

Additional terms relating to arrays and the hybridization of nucleic acids to such arrays may be found, for example, in U.S. Pat. No. 6,399,394 and U.S. Pat. No. 6,410,243.

The invention provides a method for expanding the range of use of a DNase I-like enzyme. As used herein, a “DNase I-like enzyme” is a natural, recombinant or synthetic enzyme, fragment thereof, or fusion protein thereof, that substantially nonspecifically cleaves single-stranded, double-stranded, triple-stranded, or partially single-stranded, double-stranded, or triple-stranded DNA molecules, or DNA:RNA hybrids to release mono-, di-, tri- and oligonucleotide products with 5′-phosphorylated and 3′-hydroxylated ends. As used herein, “substantially nonspecific cleavage” means that variability of cleavage at a given base usually does not vary substantially from that of bovine pancreatic DNase I.

In one aspect, a DNase I-like enzyme produces single-strand nicks (e.g., in the presence of Mg²⁺). In another aspect, a DNase I-like enzyme produces double-strand nicks (e.g., in the presence of Mn²⁺ and absence of Mg ²⁺). In a further aspect, a DNase I-like enzyme comprises bovine pancreatic DNase I, EC:3.1.21.1 or an enzyme which comprises substantially the same specific activity of bovine pancreatic DNAse I. In one aspect, the DNase I-like enzyme is a recombinant enzyme. In certain aspects, a DNase I-like enzyme lacks an actin-binding domain but otherwise retains the salt sensitivity of native bovine pancreatic DNase I enzyme, e.g., loses about 50% or more activity in the presence of ≧100 mM of a monovalent salt such as NaCl or KCl.

In one embodiment, the invention provides a method of contacting a DNA molecule with a DNase I-like enzyme in the presence of a salt concentration inhibitory to the enzyme, e.g., a salt concentration in which the enzyme loses at least about 10%, at least about 20%, at least about 30%, at least about 40%, at least about 50%, or more of its activity. As used herein, the “activity” of a DNase I-like enzyme refers to a measure of ability of the DNase I-like enzyme to catalyze cleavage of a selected substrate over a selected period of time. While any of a number of assays can be used to monitor DNase I-like activity, in one aspect, an enzyme having a DNAse I-like activity has a specific activity of >10,000 units/mg, where one unit is defined as the amount of enzyme that increases the absorbance at A260 nm in a 1 cm path length at a rate of 0.001 units per min per ml of 0.05 mg/ml calf thymus DNA (Sigma) in the presence of 10 mM Tris-HCl, pH 8.0, 0.1 mM CaCl₂ and 1 mM MgCl₂ (see, e.g., Kunitz. J. Gen. Physiol. 1950;33:349-362).

In one embodiment, the method comprises contacting the DNase I-like enzyme with a DNA template (e.g., single-stranded, double-stranded, partially double-or single-stranded DNA or a DNA:RNA hybrid) in the presence of an effective amount of organic solvent to permit digestion of at least about 50% of the DNA template in 15-30 minutes to oligonucleotides of 100 bases or less, 50 bases or less, 20 bases or less, 10 bases or less, or 3 bases or less. In one aspect, the organic solvent is not glycerol, although glycerol may be added as an additional organic solvent.

In one aspect, the organic solvent comprises an alcohol which is not glycerol, although glycerol may be provided as an additional alcohol. Exemplary alcohols include, but are not limited to low molecular weight alcohols, such as monohydroxyl alcohols, e.g., methanol, ethanol, isopropanol (e.g., 1- and 2-isopropanol) and butanol (e.g., 1- and 2-butanol). Other examples include, but are not limited to, di-hydroxylic alcohols, such as ethane diol, propane diol, butane diol, and the like. Still other examples include tri-hydroxylic alcohols.

In one aspect, an effective amount of an organic solvent comprises greater than approximately 20% v/v organic solvent, greater than about 45% v/v organic solvent, greater than about 50% v/v organic solvent, and up to about 99% v/v organic solvent. In one aspect, the DNase I-like enzyme retains its activity in the presence of at least about 10 mM of a monovalent salt such as NaCl or KCl, at least about 20 mM, at least about 30 mM, at least about 50 mM, at least about 100 mM, at least about 150 mM, or at least about 200 mM of the monovalent salt. At lower volumes of organic solvent, the lowest molecular weight alcohols may be preferred (e.g., such as methanol or ethanol).

The remainder of the solution in which DNase digestion takes place may comprise any standard buffer, e.g., comprising appropriate monovalent and/or divalent cations. In one aspect, a 1× DNase I digestion buffer comprises 10 mM Tris-HCl, pH 8.0, 1 mM MgSO₄, and 1 mM CaCl₂.

In certain aspects, the digestion buffer does not comprise a divalent cation such as Mg²⁺ or Ca²⁺.

DNA digestion can be performed in a variety of applications, e.g., to remove contaminating genomic DNA from an RNA sample, to degrade a DNA template in a transcription reaction, in a nick translation reaction or DNase I footprinting reaction. Methods for performing these techniques are known in the art.

The average size of the resulting DNA fragments generated by the method can be modulated by optimizing enzyme to substrate ratios and incubation time to suit a desired application.

In one embodiment, the invention further relates to a method comprising treating a sample of RNA to remove a substantial amount of gDNA while maintaining RNA integrity in a sample. In one aspect, the method comprises isolating RNA. In another aspect, the method comprises isolating RNA in a potentiating amount of organic solvent in an aqueous buffer (e.g., from 1% to 80% v/v aqueous buffer), e.g., an amount that results in optimal recovery of RNA compared to recovery in the absence of organic solvent and the presence of 100% aqueous buffer.

In one embodiment, a sample is homogenized in an extraction buffer. Sample sources include, but are not limited to, animals, plants, fungi (e.g., such as yeast), bacteria, and portions thereof. In one aspect, the animal can be a mammal, and in a further aspect, the mammal can be a human. Sample sources may additionally include virally infected cells, as well as transgenic animals and plants or otherwise genetically modified animals and plants. In addition, the sample can originate from experimental protocols, for example, from a polymerase chain reaction or from the products of an enzymatic reaction (e.g., a polymerization and/or transcription reaction).

In certain embodiments, samples are lysed before, during, or after homogenization. Suitable lysis solutions are known in the art. However, in one aspect, the lysis solution comprises a chaotropic salt, and/or additives to protect nucleic acids in the sample from degradation or reduced yield. Suitable salts include but are not limited to, urea, formaldehyde, ammonium isothiocyanate, guanidinium isothiocyanate, guanidinium hydrochloride, formamide, dimethylsulfoxide, ethylene glycol, tetrafluoroacetate, diamineimine, ketoaminimine, hydroxyamineimine, aminoguanidine hydrochloride, aminoguanidine hemisulfate, hydroxylaminoguanidine hydrochloride, sodium iodide, sodium perchlorate, and mixtures thereof. In another aspect, the lysis solution comprises one or more enzymes to facilitate disruption of cells in a sample. Suitable enzymes include, but are not limited to, a protease, lysozyme, zymolase, cellulase, and the like. In still other aspects, a lysis solution may include one or more agents for stabilizing nucleic acids, such as, but not limited to cationic compounds, detergents (e.g., SDS, Brij, Triton-X-100, Tween 20, DOC, and the like), chaotropic salts, ribonuclease inhibitors, chelating agents, DEPC, vanadyl compounds, and mixtures thereof. Examples of ribonuclease inhibitors can be found in Farrell R. E. (ed.) (RNA Methodologies: A Laboratory Guide for Isolation and Characterization, Academic Press, 1993) and Jones, P. et al. (In: RNA Isolation and Analysis, Bios Scientific Publishers, Oxford, 1994). In one aspect, RNAlater® (Ambion Inc., Austin, Tex., U.S. Pat. No. 6,204,375) is used as an RNAse inhibitor. In one embodiment, a lysis solution comprising at least about 4M guanidine isothiocyanate (e.g., from about 4M to about 6M) guanidine isothiocyanate is used in a Tris buffer of from about pH 6-8 (e.g., about pH 6.6 to about 7.5), EDTA (e.g., about 10 mM) and optionally, about 0.5-1% β-mercaptoethanol is used.

In still another aspect, the lysis solution comprises an amount of salt, which is typically inhibitory to the activity of a DNase I-like enzyme, e.g., at least about 10 mM of a monovalent salt such as NaCl or KCl, at least about 20 mM, at least about 30 mM, at least about 50 mM, at least about 100 mM, at least about 150 mM, or at least about 200 mM of the monovalent salt.

Mechanical homogenization can be performed using methods known in the art, e.g., such as by using a rotor-stator homogenizer, such as by grinding in a mortar and pestle with liquid nitrogen, mechanical disruption with a tissue homogenizer, such as a Polytron® or Omniprobe® homogenizer, manual homogenization (e.g., with a Dounce homogenizer), shaking the sample in a container with metal balls, or vortexing vigorously. Additionally, or alternatively, samples can be homogenized by ultrasonic disruption. In one aspect, homogenization is done in a high chaotrope concentration solution effectively lysing cells and destroying cellular enzymatic activity, such as the activity of nucleases, until a desired nuclease can be added under controlled conditions.

In one aspect, a homogenized sample is transferred to a device according to the invention for contacting with a separation module which preferentially retains genomic DNA and cellular debris while allowing RNA molecules to pass through.

As used herein, the term “module” refers to an element or unit in the device that may or may not be removable from the device. In one aspect, the device comprises a housing having an open end and comprises walls defining a lumen into which the module fits. In another aspect, the device comprises a closed bottom end. The separation module may be removable from the housing or an integral part of the housing or some combination thereof. The shape and dimensions of the housing may vary. However, in one embodiment, the housing is shaped like a tube or column. In another aspect, the housing is shaped like a tube and the separation module is provided in the form of a column that fits into the tube, the remaining space defining a collection compartment or chamber for receiving flow through from the separation module.

In certain aspects, a plurality of device housings is provided in a holder or container or rack and a plurality of separation modules (e.g., columns) may be inserted into the lumen of each of the housings. In one aspect, the plurality of device housings is provided as a single unit (e.g., molded as a single unit from a plastic or other suitable material) comprising a plurality of lumens for receiving a plurality of columns.

Individual separation modules may be separated from each other one at a time, e.g., by unscrewing or snapping apart. Likewise, the housing may be made from a variety of materials, including but not limiting to, a polymeric material such as plastic, polycarbonate, polyethylene, PTFE, polypropylene, polystyrene and the like.

In one embodiment, the separation module separates two different types of biopolymers from each other. In one aspect, the separation module separates DNA (such as genomic DNA) from RNA (e.g., such as total cellular RNA). In another aspect, the separation module comprises one or more filters or layers of beads or other type of matrix. For example, in one aspect, the separation module comprises a porous material. Suitable materials for fabricating the module include, but are not limited to, glass fibers or borosilicate fibers, silica gels (which may be further treated using chaotropic salts), polymers (e.g., beads, filters, membranes, fibers) and the like.

In one aspect, the separation module comprises a fiber material that demonstrates particle retention in the range of about 0.1 μm to about 10 μm diameter equivalent. The fibers can have a thickness ranging from about 50 μm to about 2,000 μm. For example, in one aspect, a fiber filter has a thickness of about 500 μm. The specific weight of a fiber filter can range from about 75 g/m² up to about 300 g/m2. Multiple fiber layers are envisaged to be within the scope of this invention. The fiber may, optionally, comprise a binder, e.g., for improving handling of the fiber or for modifying characteristics of a composite fiber (i.e., one which is not pure borosilicate). Examples of binders include, but are not limited to, polymers such as acrylic, acrylic-like, or plastic-like substances. Although it can vary, typically binders may represent about 5% by weight of the fiber filter.

The pore size of the filter may be uniform or non-uniform. Where a plurality of filters are used, the pore size of each filter may be the same or different. In another aspect, suitable pore sizes may range from about 0.1 μm to about 2 mm.

In a particular aspect of this invention, the separation module comprises at least one layer of fiber filter material along with a retainer ring that is disposed adjacent to a first surface of the fiber filter material that securely retains the layer(s) of fiber filter material so that they do not excessively swell when sample is added. In one aspect, a frit is provided which is disposed adjacent to a second surface of the fiber filter material. The frit may assist in providing support so that the materials of the filter fibers do not deform. In one aspect, the frit is composed of polyethylene of about 90 μm thick. In certain aspects, the separation module comprises at least two layers of filter material, at least three layers, at least four layers, at least five layers, at least six layers, at least seven layers, at least 8 layers, at least 9 layers, or at least 10 layers.

In one embodiment, the separation module comprises Whatman GF/F Glass Fiber Filters (cat no. 1825-915) (available from Fisher Scientific, Atlanta, Ga.) or an eq equivalent material. Multiple layers (of the large sheets or disks supplied) may be punched, for example, with a 9/32″ hand punch (McMaster-Carr, Chicago, Ill.) and placed into a spin column (Orochem, Westmont, Ill.) fitted with a 90 μm polyethylene frit (Porex Corp., Fairburn, Ga.) on which the fibers may rest. The filter materials may be secured in the column with a retainer ring on top of the filter materials to prevent excessive swelling of the fibers or movement during centrifugation. In one aspect, the separation module that is used is the prefiltration column available in Agilent's Total RNA Isolation Mini Kit prefiltration column (Catalog #5185-6000) from Agilent Technologies, Inc. (Palo Alto, Calif.).

In one aspect, the separation module does not comprise a matrix for anion exchange.

Flow-through from the column comprising RNA molecules, obtained after centrifugation or application of pressure to the device, is collected within the collection module of the device. In one aspect, a sample is applied to the separation module in a solution comprising a chaotropic agent and an organic solvent, such as an alcohol, in the range of about 40-60% by volume. As discussed above, exemplary alcohols include, but are not limited to, monohydroxyl alcohols, e.g., methanol, ethanol, isopropanol (e.g., 1- and 2-isopropanol) and butanol (e.g., 1- and 2-butanol). Other examples include, but are not limited to, di-hydroxylic alcohols, such as ethane diol, propane diol, butane diol, and the like. In another aspect, a DNAse I-like enzyme is added to the solution in suitable quantities to convert greater than about 50%, greater than about 60%, greater than about 70%, greater than about 80%, greater than about 90%, or greater than about 95% of genornic DNA in the sample to fragments of 20 bp or less, e.g., 0.2-200 units per μg of DNA.

In a certain aspects, sample is applied to the separation module and the separation module is washed with a solution comprising an organic solvent in the range of about 50-100% v/v. However, in another aspect, the organic solvent is provided in a potentiating amount to provide for optimal recovery of RNA from a sample being treated with the DNase I-like enzyme. In still other aspects, the aqueous component of the wash solution comprises a concentration of a salt which is typically inhibitory to a DNAse I-like enzyme, e.g., at least about 10 mM of a monovalent salt such as NaCl or KCl, at least about 20 mM, at least about 30 mM, at least about 50 mM, at least about 100 mM, at least about 150 mM, or at least about 500 mM of the monovalent salt. In a further aspect, a DNase I-like enzyme is added to the solution in a suitable quantity as described above.

In alternative, or additional aspects, the solid phase material within the separation module is impregnated with a DNAse I-like enzyme (e.g., in a lyophilized or dried form) and can be activated by contacting a sample to the solid phase material in a solution comprising at least about 40% organic solvent, as described above. In certain aspects, the solution additionally comprises a chaotropic salt.

RNA in the flow-through from the separation module can be collected within the lumen of the housing between the module and the closed end of the same or a different device (i.e., the separation module can be transferred to the housing of a different device). This portion of the device forms the “collection module.” RNA collected in the collection module can be removed from the collection module for further processing steps. Additionally, or alternatively, processing steps may occur in the collection module. While there may generally be sufficient organic solvent and salt in the wash solution or added to the lysis solution to precipitate RNA as it is passing through the separation module, additional organic solvent may be added in the collection module, e.g., to wash a pelleted RNA sample or further enhance the precipitation process.

In one aspect, the separation module is provided in the form of a column that fits into the lumen defined by the walls of the device housing and the collection module is formed in the space between the column and the closed bottom end of the housing. Removing the column from the device provides access to the collection module. Alternatively, the collection module may be removed from the device (e.g., by snapping off or twisting). In one aspect, the closed bottom end may comprise a cap or cover which may be removed to obtain collected RNA-enriched material.

In still other embodiments, a flow through from a separation module is collected in a collection module and transferred to a new collection module which comprises molecules (e.g., in the form of a membrane, matrix, gel, particles, beads, filter, and the like) for specifically binding RNA or for capturing or trapping RNA (e.g., such as precipitated RNA), for example, to remove any remaining contaminants in the solution or to further concentrate a sample. For example, an RNA capture membrane may be provided as part of the collection module to facilitate the collection of the RNA precipitate, washing of the collected precipitate (reducing wash volumes and centrifugation times) and re-suspension and elution or release of RNA. Alternatively, the flow through may be collected directly in a collection module, which comprises the RNA-binding molecules or other RNA-capture material (e.g., such as a matrix for trapping precipitated RNA).

In certain aspects, the collection module includes material that reversibly captures RNA. Suitable nucleic acid capture materials are known in the art and include, but are not limited to, SiO₂-based materials or silicon carbide (see, e.g., U.S. Pat. Nos. 6,177,278 and 6,291,248). As an alternative to silicon carbide, silica materials such as glass particles, glass powder, silica particles, glass microfibers, diatomaceous earth, and mixtures of these compounds may be employed. Nucleic acid capture materials may be combined with chaotropic salts to isolate nucleic acids. In one aspect, a nucleic acid capture material comprises a silicon carbide matrix, e.g., such as silicon carbide fibers or whiskers. In another aspect, the capture materials comprise silica carbide whiskers which comprise a comparatively high specific surface area material, greater than about 0.4 m²/g, greater than 1 m²/g, greater than 2 m²/g, greater than 3 m²/g or about 3.9 m²/g as measured by surface Nitrogen absorption.

In another aspect, the collection module comprises one or more polymeric membranes, examples of which include, but are not limited to, polysulfone, e.g., such as a BTS membrane (Pall Life Sciences), PVDF, nylon, nitrocellulose, PVP (poly(vinyl-pyrrolidone)), MMM filters (Pall Life Sciences, available from VWR, Pittsburg, Pa.) and composites thereof. In one aspect, the membrane is a composite of Polysulfone and PVP. In another aspect, the binding material comprises an asymmetric membrane with pores that gradually decrease in size from the upstream side to the downstream side. In one aspect, the membrane comprises pore sizes from about 0.1 μm to 100 μm. In another aspect, the membrane comprises pore sizes of from about 0.1 μm to 5 μm, or from about 0.1 μm to about 10 μm, or from about 0.4 μm to about 0.8 μm. In still another aspect, the binding material comprises a hydrophobic and/or hydrophilic material. Glass fiber filters, such as used in the separation module can also be used.

In one embodiment of the present invention, the collection module comprises an isolation column comprising an inlet and an outlet between which lies a chamber comprising a single or multiple layers of a polymeric membrane, examples of which include polysulfone, PVP (Poly(vinylpyrrolidone)), MMM membrane (Pall Life Science), BTS, PVDF, nylon, nitrocellulose, and composites thereof. A retainer ring and a frit can be disposed about the membrane(s) to retain them within the collection module. For example, a retainer ring may be disposed proximal to the inlet while a frit may be disposed proximal to the outlet.

In one aspect, the column comprises an asymmetric porous membrane comprising of polysulfone and polyvinylpyrrolidone. In one aspect, the membrane comprises a first surface and a second surface, the first surface having pores which are larger than the pores on the second surface. For example, in one aspect, the first surface has 30-40 μm diameter pores and the second surface has 0.1-0.10 μm diameter pores, or 0.4-0.8 μm diameter pores. In another aspect, the membrane comprises intermediate sized pores between the first and second surface. In still another aspect, the larger diameter pores are on the upper side of the membrane while the smaller diameter pores (proximal to the collection module of the device) are on the lower surface.

In one aspect, the matrix or membrane is substantially insoluble at elevated pHs and reversibly absorbs nucleic acids. In another aspect, the matrix is an MMM membrane or plurality of MMM membranes.

Examples of RNA capture materials additionally include, but are not limited to, various types of silica, including glass and diatomaceous earth. In some aspect, binding materials include binding moieties stably associated with a solid phase, such that RNA molecules will bind to the solid phase by virtue of this association. RNA-capture materials include cation exchange groups such as carboxylates, and hydrophobic interaction groups. Thus, examples of solid phase nucleic acid capture materials also include silica particles, magnetic beads coated with silica, and resins coated with cation exchange groups, hydrophobic interaction groups, dyes, and the like. However, in a further aspect, the RNA capture material does not comprise silica.

In certain aspects, the RNA capture material comprises a porous or semi-porous of fibrous material which captures precipitated RNA within its pores/between its fibers. It should be noted that an RNA capture material also may comprise an RNA-binding material and that the mechanism by which RNA is selectively retained within the capture material is not a limiting feature of the invention.

Although in one aspect, the separation module substantially removes all of genomic DNA in a sample, in certain aspects, DNase I-like enzymes are additionally added to the collection module, e.g., in solution or in impregnated in an RNA-capture material such as described above. In certain aspects, digestion by a DNAse I-like enzyme within the collection module occurs in the presence of an at least about 40% v/v solution of organic solvent as described above.

In still other aspects, however, a cell or tissue lysate is contacted to the separation module in a less than 20% solution of organic solvent, such that RNA is not precipitated as it passes through the separation module. RNA can be precipitated and additionally treated with a DNAse I-like enzyme in an at least about 20% solution of organic solvent within the collection module using RNA-capture materials as described above. In still other aspects, it is desirable not to add a DNase-I like enzyme to the separation module, e.g., where the separation module is later used to collect genomic DNA, for example, in methods for obtaining both RNA and genomic DNA in a sample.

RNA eluted or released from RNA-capture materials in the collection module can be precipitated (e.g., in an amount of solvent which further comprises a DNAse I-like enzyme) and pelleted by centrifugation (e.g., a spin step of 30 seconds at room temperature at 16,000 g). Pelleted nucleic acids may be resuspended, for example, after washing at least once, or at least twice, with a wash solution, for example, such as 25 mM Tris-HCl pH 7.5, 80% ethanol. After a final wash, pelleted nucleic acids are resuspended in a suitable buffer, for example, H₂O or TE.

The quality and/or quantity of nucleic acids collected may be evaluated and optimized using methods well known in the art, such as obtaining an A260/A280 ratio, evaluating an electrophoresed sample, or by using Agilent Technologies® RNA 6000 Nano assay (part no. 5065-4476) on the Agilent Technologies® Bioanalyzer 2100 (part no. G2938B, Agilent Technologies, Inc., Palo Alto, Calif.) as per manufacturer's instructions.

As discussed above, in addition to RNA isolation, organic solvent/aqueous solutions according to the invention can be used with DNase I-like enzymes in a variety of applications.

In one embodiment, a method according to the invention comprises providing a DNA template encoding an RNA product and contacting the DNA template with an RNA polymerase in the presence of suitable amounts of ribonucleotides under conditions for performing an in vitro transcription reaction. The remaining DNA template is removed by contacting the solution with an amount of organic solvent to produce a solution that is suitable for maintaining the activity of a DNAse I-like enzyme despite the presence of an amount of salt that is typically inhibitory to that DNase I-like enzyme. In one aspect, the solution after contacting with organic solvent comprises at least about 20% v/v organic solvent and the DNA template is incubated in the solution for a suitable amount of time (e.g., 10-15 minutes at 25° C. to about 37° C., or higher, e.g., if using a thermostable DNase I-like enzyme). RNA transcripts may be collected by centrifugation, optionally, after adding additional amounts of organic solvent. In one aspect, RNA transcripts are contacted to an RNA-binding matrix, such as described above.

In another embodiment, an organic solvent/aqueous solution according to the invention is used in a nick-translation reaction to label a DNA molecule. In one aspect, the method comprises providing a DNA template and a DNase I-like enzyme in the presence of at least about 40% of an organic solvent (v/v) as described above and incubating the enzyme under conditions for introducing nicks into the DNA template. In another aspect, the aqueous component of the solution comprises an amount of salt that is typically inhibitory of the DNase I-like enzyme. Nicked DNA is then precipitated and contacted with deoxyribonucleotides, a DNA polymerase such as E. coli DNA polymerase I, and ligase (e.g., such as T4 ligase), resuspended in buffer and incubated under conditions suitable for DNA polymerization of the nicked template. In certain aspects, the DNAse-I like enzyme is inactivated prior to precipitation, e.g., by the addition of additional solvent, by the addition of EDTA and/or by heating the enzyme (e.g., at 70° C. for about 5 minutes). Nick-translated and ligated DNA can be separated from unincorporated dNTPs using methods known in the art, e.g., by chromatography through a column of Sephadex G-50 or by spun-column chromatography.

In a further embodiment, an organic solvent/aqueous solution according to the invention is used in location analysis. In one aspect, proteins that bind genomic DNA (e.g., such as proteins in a cell) are crosslinked to the DNA, e.g., by formaldehyde or another suitable fixative or condition. In certain aspects, the proteins are predefined, e.g., one or more known proteins are added in vitro to a solution of DNA. In other aspects, the proteins are from a complex sample, such as a cellular lysate. The resulting mixture, which includes DNA bound by protein and DNA which is not bound by protein is exposed to a DNase I-like enzyme in an organic solvent/aqueous solution at a final concentration which is at least about 20% v/v organic solvent for a sufficient amount of time to generate DNA fragments, including some which are bound by protein. Unbound DNA, digested to sizes of about 20 bases or less by the DNAse I-like enzyme can be removed, e.g., via a spin column.

Protein-DNA complexes can be contacted with protein-binding molecules, optionally, after pelleting by centrifugation and resuspending the complexes in an appropriate buffer for sorting particular protein-DNA complexes. Alternatively, complexes can be sorted directly in organic solvent-containing buffer.

Suitable sorting methods include, but are not limited to, immunoprecipitation or affinity-based methods which comprise the use of predefined protein-binding molecules (e.g., antibodies, affibodies, aptamers and the like) stably associated with a solid support. Crosslinked proteins may subsequently be removed from DNA, e.g., by heating at a temperature that also inactivates the DNase I-like enzyme, and the remaining fragments can be detected by a suitable method to identify the genomic region to which the proteins bind. For example, fragments can be sequenced or applied to an array for binding to a nucleic acid probe which can be used to identify and characterize the fragment as described in U.S. Pat. No. 6,410,243. In certain aspects, fragments are amplified prior to application to an array, e.g., by a substantially unbiased amplification method such as multiple-strand displacement amplification or through the use of primer binding sites ligated to the ends of the fragments as described in U.S. Pat. No. 6,410,243.

Generally, a DNase I-like enzyme can be contacted to a DNA template in an organic solvent/aqueous solution according to the invention for use in any application in which a DNase I-like enzyme is used. The applications described above are not limiting and others will be obvious to those of skill in the art based on the disclosure herein and are encompassed within the scope of the invention.

In an additional embodiment, the invention further relates to storage-stable solutions of a DNase I-like molecule comprising a DNase I-like enzyme in an about 20% or greater v/v solution of an organic solvent (including up to about 100%) which is not glycerol, though glycerol may be added as an additional component of the solution. Before use, an aqueous solution comprising sufficient water or buffer to produce an at least about 20% to 99% v/v solution of organic solvent may be added along with a suitable template for digestion of the template as described above. In certain aspects, a sufficient amount of water to provide a potentiating amount of organic solvent for isolating RNA from a sample is provided. In one aspect, the DNase I-like enzyme is lyophilized or otherwise dehydrated prior to contacting with the organic solvent.

In further embodiments, the invention relates to kits comprising a DNase I-like molecule, an organic solvent and, optionally, an aqueous solution. In one aspect, the organic solvent comprises an alcohol, which can include, but is not limited to, a monohydroxyl alcohol, e.g., methanol, ethanol, isopropanol (e.g., 1- and 2-isopropanol) and butanol (e.g., 1- and 2-butanol), a di-hydroxylic alcohol, such as ethane diol, propane diol, butane diol, and the like, or a combination thereof. In one aspect, the organic solvent and aqueous solution are mixed to provide a final volume which is at least about 20% to about 99% of organic solvent. In another aspect, the organic solvent is present at a higher concentration, e.g., up to about 100% v/v, and can be diluted by an aqueous solution, which is optionally included in the kit. In certain aspects, the DNase I-like enzyme is provided in an organic solvent, e.g., in a storage-stable form, as described above, or is provided in a ready-to-use form, e.g., in the presence of an amount of aqueous solution that permits DNA digestion. In certain aspects, the aqueous solution comprises an amount of salt that is typically inhibitory to the DNase I-like enzyme. In still other aspect, the kit comprises a device comprising a separation and/or collection module as described above. In certain aspects, the separation and/or collection module comprise a solid phase, which is impregnated with a DNase I-like enzyme and, optionally, an organic solvent.

### EXAMPLE

The invention is demonstrated further by the following illustrative example which illustrates the invention but is not intended to limit its scope.

DNase I-like Activity Determinations.

The enzymatic hydrolysis of calf thymus genomic DNA was assayed using a method modified from that described by Desai and Shankar (Eur. J. Biochem., 2000;267; 5123-5135). The standard reaction mixture was 0.1 mL volume, containing 30 μg of sonicated native calf thymus genomic DNA (Sigma, St. Louis Mo., #D-3664), in a buffer solution composed of 100 mM TrisHCl(pH 8.0)/10 mM MgSO4/1 mM CaCl2, with appropriately diluted DNase (usually 0.02-1.0 Enzyme Units, as defined below). The reaction was initiated by the addition of enzyme, with incubation at room temperature for a defined period of time (usually 10-20 minutes), after which the reaction was terminated by rapid sequential addition of 0.1 mL of 1 mg/mL Bovine Serum Albumin (Sigma, #A3803) and 1.0 mL of ice cold 2% (v/v) Perchloric Acid. The terminated reaction mixture was vortex mixed, then chilled on ice for 20-30 minutes, follwed by centrifugation at 16,000×g for 10 minutes at 4 degrees centrigrade. The clarified supernatant contains acid-soluble oligonucleotides liberated by the action of DNase I-like activity, at concentrations determined using absorbance measurements determined in a 1 cm pathlength cell in the Agilent 8453 spectrophotometer (Agilent Technologies, Wilmington Del.). A molar extinction coefficient of 10,000 M⁻¹ cm⁻¹ was employed for oligonucleotide concentration estimation in the acidic solution. Unit activity under these assay conditions is defined as umol of acid-soluble oligonucleotides generated per minute at 25 degrees centigrade. In the experiments shown in the examples that follow, bovine pancreatic DNase I is employed to illustrate the effects of various manipulations on the activity of this enzyme. Typically, the reaction employs approximately 0.1 enzyme units, as defined by the Kunitz assay, as described above (see Kunitz. J. Gen. Physiol. 1950;33:349-362).

### Example 1

### Isolation of RNA

During the isolation of RNA from small samples of biological origin, manipulations involving the sample should be kept to a minimum, and since the quantities of RNA may be in the nanogram range, all manipulations should be conducted in such a way to reduce loss of the mass and physical integrity of RNA. An optional DNase I digestion step is often used to remove gDNA from RNA samples, thereby removing the DNA as a contaminant, improving the purity and experimental relevance of the isolated sample. DNAase I digestion should therefore be conducted considering the desire to minimally manipulate the RNA sample, and to reduce the opportunity to lose the RNA. In the description that follows, we demonstrate that the DNase I digestion of gDNA contaminants can be conducted in such a manner to prevent solubilization of RNA, and concomitant loss from an RNA-collection device, while selecting conditions which are shown to permit highly active DNase activity.

RNA Isolation Method.

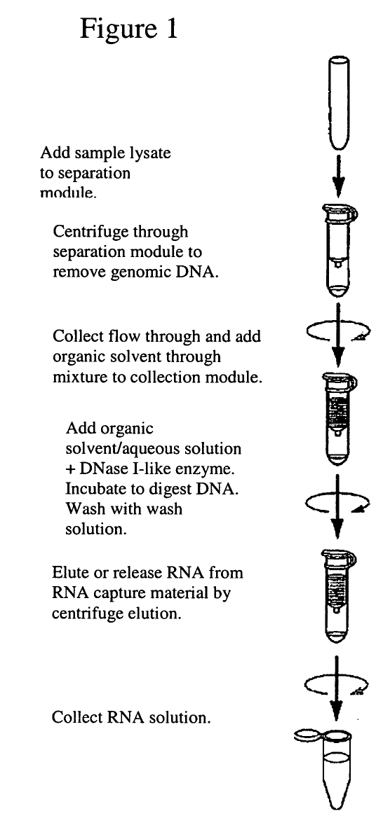

RNA was isolated using a device and protocol as shown in FIG. 1. Tissue or cell homogenate was placed in 100 μl of Lysis Solution (4M guanidine isothiocyanate, 25 mM Tris pH7.5, 10 mM EDTA, 1% β-mercaptoethanl). The sample was homogenized using a conventional rotor-stator homogenizer with a stainless steel probe at 15,000 rpm. Up to 200 μl of homogenate (equivalent to 500,000 of cells and 2.5 mg of tissue) was centrifuged through a micro-prefiltration column available in Agilent's Total RNA Isolation Micro-Kit (Product No. unknown at this time) from Agilent Technologies, Inc. (Palo Alto, Calif.) serving as a separation module for 3 minutes at full speed (for a typical microcentrifuge, approximately 16,000×g). The flow through was saved for RNA isolation.

A volume of organic solvent was added to the flow through to produce an at least 35% v/v solution of organic solvent and the solution was contacted to an RNA capture material in the form of a column in a collection tube as shown in FIG. 1. Flow through from this column was discarded after centrifugation for 30 seconds at full speed. The RNA-loaded column was replaced in the collection tube and bovine pancreatic DNase I in an appropriate solution was added (hereafter referred to as the “DNase Solution”). The DNase Solution and enzyme was left in contact with the RNA isolation membrane within the device during incubation at room temperature for a specified period of time, usually between 5 and 15 minutes. Following DNase I digestion, the enzyme and DNase Solution are removed by addition of 500 μl of a wash solution, then centrifugation for 30 seconds at full speed. The flow-through was discarded and 80% ethanol in dilute buffer was added to the micro-isolation column, which was centrifuged for 30 seconds at full speed. Flow through was again discarded and the micro-isolation column is then spun for 2 minutes at full speed to completely remove trace amounts of wash solution and to remove ethanol. The micro-isolation column is transferred into a new 1.5 ml RNase-free final collection tube. 15-30 μl of RNase-free water is added to the top center of membrane (without touching the membrane). After 1 minute, the column in the final collection tube is centrifuged for 1 minute at full speed to collect RNA from the isolation column.

RNA Assay Methods.

RNA quantities were determined by solution phase assay in a 96 well plate format using a highly sensitive fluorimetric method supplied in the RiboGreen RNA Quantitation Kit from Molecular Probes (Eugene, Oreg.) with minor modifications, as follows; the concentrated Dye reagent is diluted 1/4000 final concentration in TE buffer for use in the assay, and the concentration range of RNA standards to construct the calibration curve was set at 0.25 ng to 12.5 ng per 250 μL final assay volume. Fluorescence measurements were conducted using the Perkin-Elmer LS-55 instrument (Groton, Conn.) with excitation at 490 nm and emission at 535 nm. All samples were measured in duplicate. RNA integrity and estimation of quantities were also conducted using the Agilent 2100 BioAnalyzer microfluidic system (Agilent Technologies, Inc, Wilmington, Del.) using the PicoAssay method.

Genomic DNA contamination was quantified using a 5′ nuclease assay, or “real-time” PCR assay, run on the Applied Biosystems Prism 7000 Sequence Detection System (Applied Biosystems, Foster City, Calif.). This type of assay monitors the amount of PCR product that accumulates with every PCR cycle. Isolated tcRNA (˜4 ng) from HeLa S3 cells was added to a re action mixture containing primers and probe specific for human (Genbank Accession NM-002046) glyceraldehydes-3-phosphate dehydrogenase (GAPDH). All samples were run in a reaction mixture consisting of both primers at 500 nM, fluorescent probe at 200 nM, and 1× Taqman Universal Master Mix (part#43044437) in conditions well know to those skilled in the art. Serial dilutions of human genomic DNA (Promega, Madison, Wis.) were used for the generation of a standard curve. All samples, standards and no-template controls were run in duplicate.

The human GAPDH assay amplified a 69 base-pair fragment within an exon. The GAPDH assay primers and probe were designed using the Primer Express software package (Applied Biosystems, Foster City, Calif., Part no. 4329442). The primers were desalted and the probe (5′ labeled with 6-FAM and 3′ labeled with BHQ-1) was purified by anion exchange followed by reverse phase HPLC (Biosearch Technologies, Novato, Calif.).

The assay cycling parameters for both assays were the default conditions set by the manufacturer, i.e. 50° C. for 2 min., 95° C. for 10 min., then 40 cycles of 95° C. for 15 sec. to 60° C. for 1 min. Quantification of gDNA in the isolated tcRNA was calculated from the human gDNA standard curve.

Isolation of RNA from Small Samples of HeLa Cells.

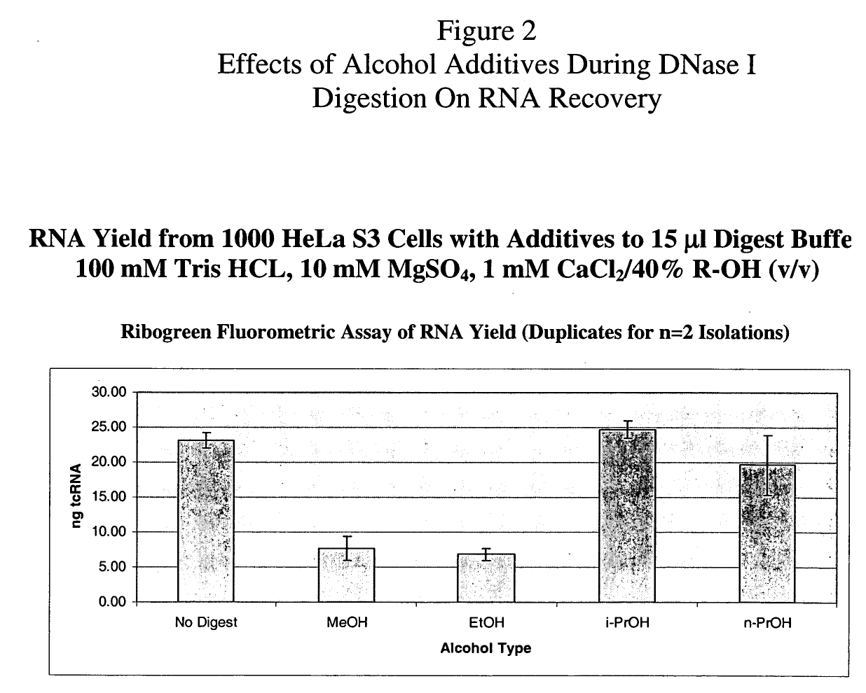

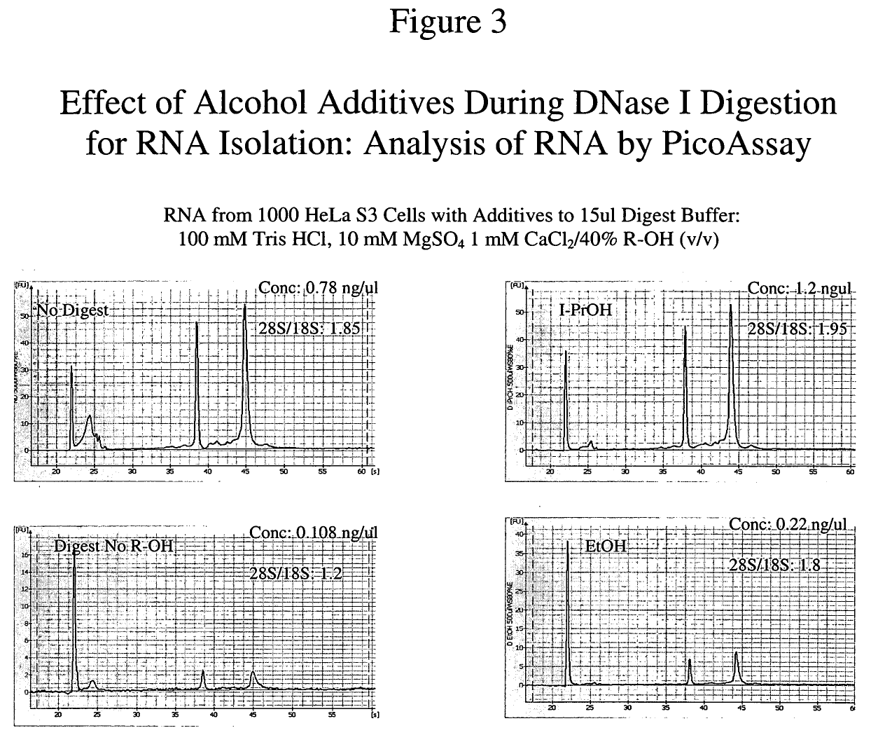

RNA was isolated from samples of 1000 HeLa S3 cells using the lysis and extraction methods described above. During the isolation, while the RNA was on the RNA collection membrane within the spin-column device, DNase I digestion was conducted “on column”, using 10 units of enzyme in 100 mM Tris HCl pH 8.0, 10 mM MgSO₄, 1 mM CaCl₂, with the addition of various alcohols to the digestion reaction, at a final concentration of 40% alcohol (v/v). Under these conditions, without the addition of alcohol to the digestion buffer, recoveries are profoundly reduced, generally by a factor of 5-10 fold below the values obtained without DNase I digestion (see for example, FIG. 3). In FIG. 2, the “No Digest” sample refers to the elimination of the Digestion Solution and DNase I altogether. Although such sample may be contaminated with several percent of gDNA, this level does not typically interfere with the RiboGreen assay measurement for RNA. This control sample represents the maximal RNA that can be recovered using this methodology. As shown in FIG. 2, the recovery of RNA from these small samples is strongly a function of the type of alcohol added to the DNase I digestion. Based on the results shown in FIG. 2, comparing the data for 40% organic solvent addition, i-propanol is the best choice for recovery, with yield results as good as those obtained without DNase I treatment, whereas methanol and ethanol are not as good choices at 40% for optimizing recovery of RNA. As shown in FIG. 3, DNase I digestion conducted under the preferred conditions described herein result in no compromise in the physical integrity of the RNA, as evidenced by clear rRNA bands in the electropherograms, with area ratios of 28S/18S rRNA approaching 2, also consistent with the isolation of high quality RNA.

The results obtained using both the RiboGreen solution phase fluorometric assay and BioAnalyzer PicoAssay are consistent in demonstrating the high recovery and integrity of RNA obtained using preferred alcohol choice at a concentration of alcohol known to readily support DNase I digestion activity. The gDNA contents in the samples shown in FIG. 3 were assayed using a quantitative PCR method as described above. In the No Digest Sample, gDNA was present at about 6 pg DNA/ng RNA, whereas with all samples subjected to DNase I digestion, no gDNA was detectable (less than 0.5 pg gDNA/ng RNA). The use of the DNase digestion step is clearly useful for reduction of gDNA contamination, in agreement with previous results referenced in this application, and by the use of alcohol addition, can be conducted under conditions that permit greatly improved recovery of RNA, particularly from biological samples containing small amounts of RNA.

Although DNase I digestion may reduce yield of RNA, the purity of RNA is increased and the addition of organic solvent increases yield compared to the treatment of sample with DNase I in the absence of organic solvent.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising a DNase I-like enzyme and an organic solvent which is not glycerol, and an RNAse inhibitor.

2. The composition of claim 1, wherein the organic solvent comprises an alcohol.

3. The composition of claim 1, wherein the organic solvent is present in at least about 20% v/v of a solution comprising the DNase I-like enzyme.

4. The composition of claim 1, wherein the organic solvent is present in at least about 60% v/v of a solution comprising the DNase I-like enzyme.

5. The composition of claim 1, wherein the DNase I-like enzyme comprises bovine pancreatic DNase I.

6. The composition of claim 1, wherein the DNase I-like enzymes comprises a recombinant enzyme.

7. The composition of claim 2, wherein the alcohol comprises a monohydroxyl alcohol.

8. The composition of claim 7, wherein the alcohol is selected from the group consisting of methanol, ethanol, isopropanol, n-propanol, butanol, isomers thereof, stereoisomers thereof, and combinations thereof.

9. The composition of claim 2, wherein the alcohol comprises a di-hydroxylic alcohol.

10. The composition of claim 9, wherein the alcohol is selected from the group consisting of ethane diol, propane diol, butane diol, isomers thereof, stereoisomers thereof, and combinations thereof.

11. The composition of claim 1, wherein the RNase inhibitor inhibits one or more of RNase A, RNase B, RNase C, RNase T1 and RNase 1.

12. The composition of claim 1 comprising at least about 99% organic solvent.

13. A kit comprising the composition of claim 12 and an aqueous solution provided in a separate container from the composition.

14. The kit of claim 13, wherein the aqueous solution comprises a solution which is inhibitory to the DNase I-like enzyme in the absence of organic solvent.

15. A kit of claim 13 comprising a DNase I-like enzyme and an organic solvent in separate containers.

16. The kit of claim 15, wherein the kit further comprises an aqueous solution which is optionally, in a separate container from the organic solvent.

17. The kit of claim 16, wherein the aqueous solution comprises a solution which is inhibitory to the DNAse I-like enzyme in the absence of organic solvent.

18. The kit of claim 13, wherein the RNase inhibitor is provided in a separate container.

19. The kit of claim 13, wherein the RNase inhibitor inhibits one or more of RNase A, B, C, RNase T1 and RNase 1.

20. The composition of claim 1, further comprising an aqueous solution which would be inhibitory to the DNase I-like enzyme in the absence of organic solvent.

21. The composition of claim 19, wherein the aqueous solution comprises at least about 10 mM of a monovalent salt.

22. The kit of claim 15, wherein the aqueous solution comprises at least about 10 mM of a monovalent salt.

23. The kit of claim 18, wherein the aqueous solution comprises at least about 10 mM of a monovalent salt.

24. A method, comprising:
contacting a sample comprising a DNA molecule and RNA molecule with a DNase I-like enzyme and a solution comprising an organic solvent which is not glycerol; and
collecting the RNA molecule.

25. The method of claim 24, wherein the solution comprises a salt concentration inhibitory to the DNAse I-like enzyme in the absence of the organic solvent.

26. The method of claim 25, wherein the organic solvent comprises an alcohol.

27. The method of claim 26, wherein the alcohol comprises a monohydroxyl alcohol.

28. The method of claim 27, wherein the alcohol is selected from the group consisting of methanol, ethanol, isopropanol, n-propanol, butanol, isomers thereof, stereoisomers thereof, and combinations thereof.

29. The method of claim 26, wherein the alcohol comprises a di-hydroxylic alcohol.

30. The method of claim 29, wherein the alcohol is selected from the group consisting of ethane diol, propane diol, butane diol, isomers thereof, stereoisomers thereof, and combinations thereof.

31. The method of claim 24, wherein the sample is a cell or tissue sample.

32. The method of claim 24, wherein the RNA molecule is collected by contacting the sample with an RNA capture material.

33. The method of claim 32, further comprising releasing the RNA molecule from the RNA capture material.

34. The method of claim 33, wherein the RNA capture material comprises a polymeric membrane.

35. The method of claim 24, further comprising contacting the sample to a solid phase under conditions in which genomic DNA preferentially remains associated with the solid phase.

36. The method of claim 32, further comprising contacting the sample to a solid phase under conditions in which genomic DNA preferentially remains associated with the solid phase.

37. The method of claim 36, wherein contacting to the solid phase occurs prior to contacting the sample to the RNA capture material.

38. An RNA capture material comprising a solid phase in contact with a DNase I-like enzyme and an organic solvent which is not glycerol.

39. The RNA capture material, wherein the material comprises a polymeric membrane.

40. The method of claim 24, wherein the solution comprises a potentiating amount of organic solvent which maximizes collection of RNA from the sample.

41. The composition of claim 2, wherein the alcohol is a tri-hydroxylic alcohol.

42. The method of claim 24, wherein the organic solvent comprises an alcohol which is a tri-hydroxylic alcohol.

43. The composition of claim 1, wherein the composition additionally comprises glycerol.

44. The method of claim 24, wherein the solution additionally comprises glycerol.
